SEQUENCE LISTING (36 sequences)

SEQ ID NO: 1 - protein, 5 aa, synthetic construct
  REGION 1..5: note = CDR-H1
TYGMS

SEQ ID NO: 2 - protein, 17 aa, synthetic construct
  REGION 1..17: note = CDR-H2
TISSGGSYTYYPDSVKG

SEQ ID NO: 3 - protein, 14 aa, synthetic construct
  REGION 1..14: note = CDR-H3
QGLDDNYAEWYFDV

SEQ ID NO: 4 - protein, 11 aa, synthetic construct
  REGION 1..11: note = CDR-L1
RASQSIYKNLH

SEQ ID NO: 5 - protein, 7 aa, synthetic construct
  REGION 1..7: note = CDR-L2
YASDSIS

SEQ ID NO: 6 - protein, 9 aa, synthetic construct
  REGION 1..9: note = CDR-L3
LQGFSTPWT

SEQ ID NO: 7 - protein, 5 aa, synthetic construct
  REGION 1..5: note = CDR-H1
SYAMS

SEQ ID NO: 8 - protein, 16 aa, synthetic construct
  REGION 1..16: note = CDR-H2
SISSGGSTYYPDSVKG

SEQ ID NO: 9 - protein, 7 aa, synthetic construct
  REGION 1..7: note = CDR-H3
GYDGYDY

SEQ ID NO: 10 - protein, 16 aa, synthetic construct
  REGION 1..16: note = CDR-L1
KSSQSLLDSDGKTYLN

SEQ ID NO: 11 - protein, 7 aa, synthetic construct
  REGION 1..7: note = CDR-L2
LVSKLDS

SEQ ID NO: 12 - protein, 9 aa, synthetic construct
  REGION 1..9: note = CDR-L3
WQGTHFPFT

SEQ ID NO: 13 - protein, 123 aa, synthetic construct
  REGION 1..123: note = variable heavy chain
EVQLVESGGDLVKPGGSLKLSCAASGFTFSTYGMSWVRQTPDKRLEWVATISSGGSYTYYPDSVKGRFTISRDNVKNTLYLQMSSLKSEDTAMYYCARQGLDDNYAEWYFDVWGAGTTVTVSS

SEQ ID NO: 14 - protein, 107 aa, synthetic construct
  REGION 1..107: note = variable light chain
DILLTQSPATLSVTPGETVSLSCRASQSIYKNLHWYQQKSHRSPRLLIKYASDSISGIPSRFTGSGSGTDYTLSINSVKPEDEGKYYCLQGFSTPWTFGGGTKLEIK

SEQ ID NO: 15 - protein, 115 aa, synthetic construct
  REGION 1..115: note = variable heavy chain
EVKLVESGGGLVKPGGSLKLSCAASGFTFSSYAMSWVRQTPEKRLEWVASISSGGSTYYPDSVKGRFTISRDNARNILYLQMSSLRSEDTAMYYCARGYDGYDYWGQGTTLTVSS

SEQ ID NO: 16 - protein, 112 aa, synthetic construct
  REGION 1..112: note = variable light chain
DVVMTQTPLTLSVTIGQPASISCKSSQSLLDSDGKTYLNWLLQRPGQSPKRLIYLVSKLDSGVPDRFTGSGSGTDFTLKISRVEAEDLGVYYCWQGTHFPFTFGSGTKLEIK

SEQ ID NO: 17 - DNA, 369 bp, synthetic construct
  misc_feature 1..369: note = nucleotides encoding variable heavy chain
  source 1..369: mol_type = other DNA
gaggtccagctggtggaatctgggggagacttagtgaagcctggagggtccctgaaactctcctgtgcagcctctggattcactttcagtacctatggcatgtcttgggttcgccagactccagacaagaggctggagtgggtcgcaaccattagtagtggtggtagttacacctactatccagacagtgtgaaggggcgattcaccatctccagagacaatgtcaagaacaccctgtacctgcaaatgagcagtctgaagtctgaggacacagccatgtattactgtgcaagacaggggcttgatgataactacgcggagtggtacttcgatgtctggggcgcagggaccacggtcaccgtctcctca

SEQ ID NO: 18 - DNA, 321 bp, synthetic construct
  misc_feature 1..321: note = nucleotides encoding variable light chain
  source 1..321: mol_type = other DNA
gacatcctgctgacccagtctccagccaccctgtctgtgactccaggagaaacagtcagtctttcctgtagggccagccagagtatttacaagaacctacactggtatcaacagaaatcacatcggtctccaaggcttctcattaagtatgcttctgattccatctctgggatcccctccaggttcactggcagtggatcagggacagattacactctcagtatcaacagtgtgaagcccgaagatgaaggaaaatattactgtcttcaaggtttcagcacaccgtggacgttcggtggaggcaccaagctggaaatcaaa

SEQ ID NO: 19 - DNA, 345 bp, synthetic construct
  misc_feature 1..345: note = nucleotides encoding variable heavy chain
  source 1..345: mol_type = other DNA
gaagtgaagctggtggagtctgggggaggcttagtgaagcctggagggtccctgaaactctcctgtgcagcctctggattcactttcagtagctatgccatgtcttgggttcgccagactccagagaagaggctggagtgggtcgcatccattagtagtggtggtagcacctactatccagacagtgtgaagggccgattcaccatctccagagataatgccaggaacatcctgtacctgcaaatgagcagtctgaggtctgaggacacggccatgtattactgtgcaagaggctatgatggttacgactactggggccaaggcaccactctcacagtctcctca

SEQ ID NO: 20 - DNA, 336 bp, synthetic construct
  misc_feature 1..336: note = nucleotides encoding variable light chain
  source 1..336: mol_type = other DNA
gatgttgtgatgacccagactccactcactttgtcggttaccattggacaaccagcctccatctcttgcaagtcaagtcagagcctcttagatagtgatggaaagacatatttgaattggttgttacagaggccaggccagtctccaaagcgcctaatctatctggtgtctaaactggactctggagtccctgacaggttcactggcagtggatcagggacagatttcacactgaaaatcagcagagtggaggctgaggatttgggagtttattattgctggcaaggtacacattttccattcacgttcggctcggggacaaagttggaaataaaa

SEQ ID NO: 21 - protein, 57 aa, synthetic construct
  REGION 1..57: note = signal peptide of heavy chain
ATGAACTTCGGGCTCAGCCTGATTTTCCTTGCCCTCATTTTAAAAGGTGTCCAGTGT

SEQ ID NO: 22 - protein, 972 aa, synthetic construct
  REGION 1..972: note = constan region heavy chain
GCCAAAACGACACCCCCATCTGTCTATCCACTGGCCCCTGGATCTGCTGCCCAAACTAACTCCATGGTGACCCTGGGATGCCTGGTCAAGGGCTATTTCCCTGAGCCAGTGACAGTGACCTGGAACTCTGGATCCCTGTCCAGCGGTGTGCACACCTTCCCAGCTGTCCTGCAGTCTGACCTCTACACCCTCAGCAGCTCAGTGACTGTAACCTCGAGCACCTGGCCCAGCCAGTCCATCACCTGCAATGTGGCCCACCCGGCAAGCAGCACCAAGGTGGACAAGAAAATTGTGCCCAGGGATTGTGGTTGTAAGCCTTGCATATGTACAGTCCCAGAAGTATCATCTGTCTTCATCTTCCCCCCAAAGCCCAAGGATGTGCTCACCATTACTCTGACTCCTAAGGTCACGTGTGTTGTGGTAGACATCAGCAAGGATGATCCCGAGGTCCAGTTCAGCTGGTTTGTAGATGATGTGGAGGTGCACACAGCTCAGACGCAACCCCGGGAGGAGCAGTTCAACAGCACTTTCCGCTCAGTCAGTGAACTTCCCATCATGCACCAGGACTGGCTCAATGGCAAGGAGTTCAAATGCAGGGTCAACAGTGCAGCTTTCCCTGCCCCCATCGAGAAAACCATCTCCAAAACCAAAGGCAGACCGAAGGCTCCACAGGTGTACACCATTCCACCTCCCAAGGAGCAGATGGCCAAGGATAAAGTCAGTCTGACCTGCATGATAACAGACTTCTTCCCTGAAGACATTACTGTGGAGTGGCAGTGGAATGGGCAGCCAGCGGAGAACTACAAGAACACTCAGCCCATCATGGACACAGATGGCTCTTACTTCGTCTACAGCAAGCTCAATGTGCAGAAGAGCAACTGGGAGGCAGGAAATACTTTCACCTGCTCTGTGTTACATGAGGGCCTGCACAACCACCATACTGAGAAGAGCCTCTCCCACTCTCCTGGTAAA

SEQ ID NO: 23 - DNA, 57 bp, synthetic construct
  misc_feature 1..57: note = signal peptide heavy
  source 1..57: mol_type = other DNA
atgaacttcgggctcagcctgattttccttgccctcattttaaaaggtgtccagtgt

SEQ ID NO: 24 - DNA, 972 bp, synthetic construct
  misc_feature 1..972: note = constant region
  source 1..972: mol_type = other DNA
gccaaaacgacacccccatctgtctatccactggcccctggatctgctgcccaaactaactccatggtgaccctgggatgcctggtcaagggctatttccctgagccagtgacagtgacctggaactctggatccctgtccagcggtgtgcacaccttcccagctgtcctgcagtctgacctctacaccctcagcagctcagtgactgtaacctcgagcacctggcccagccagtccatcacctgcaatgtggcccacccggcaagcagcaccaaggtggacaagaaaattgtgcccagggattgtggttgtaagccttgcatatgtacagtcccagaagtatcatctgtcttcatcttccccccaaagcccaaggatgtgctcaccattactctgactcctaaggtcacgtgtgttgtggtagacatcagcaaggatgatcccgaggtccagttcagctggtttgtagatgatgtggaggtgcacacagctcagacgcaaccccgggaggagcagttcaacagcactttccgctcagtcagtgaacttcccatcatgcaccaggactggctcaatggcaaggagttcaaatgcagggtcaacagtgcagctttccctgcccccatcgagaaaaccatctccaaaaccaaaggcagaccgaaggctccacaggtgtacaccattccacctcccaaggagcagatggccaaggataaagtcagtctgacctgcatgataacagacttcttccctgaagacattactgtggagtggcagtggaatgggcagccagcggagaactacaagaacactcagcccatcatggacacagatggctcttacttcgtctacagcaagctcaatgtgcagaagagcaactgggaggcaggaaatactttcacctgctctgtgttacatgagggcctgcacaaccaccatactgagaagagcctctcccactctcctggtaaa

SEQ ID NO: 25 - protein, 20 aa, synthetic construct
  REGION 1..20: note = signal peptide light chain
MVFTPHILGLLLFWISASTG

SEQ ID NO: 26 - protein, 107 aa, synthetic construct
  REGION 1..107: note = constant region light chain
RADAAPTVSIFPPSSEQLTSGGASVVCFLNNFYPKDINVKWKIDGSERQNGVLNSWTDQDSKDSTYSMSSTLTLTKDEYERHNSYTCEATHKTSTSPIVKSFNRNEC

SEQ ID NO: 27 - DNA, 60 bp, synthetic construct
  misc_feature 1..60: note = signal peptide light chain
  source 1..60: mol_type = other DNA
atggtattcacacctcatatccttggacttctgcttttctggatttcagcctccacaggt

SEQ ID NO: 28 - DNA, 321 bp, synthetic construct
  misc_feature 1..321: note = constant region light chain
  source 1..321: mol_type = other DNA
cgggctgatgctgcaccaactgtatccatcttcccaccatccagtgagcagttaacatctggaggtgcctcagtcgtgtgcttcttgaacaacttctaccccaaagacatcaatgtcaagtggaagattgatggcagtgaacgacaaaatggcgtcctgaacagttggactgatcaggacagcaaagacagcacctacagcatgagcagcaccctcacgttgaccaaggacgagtatgaacgacataacagctatacctgtgaggccactcacaagacatcaacttcacccattgtcaagagcttcaacaggaatgagtgt

SEQ ID NO: 29 - protein, 19 aa, synthetic construct
  REGION 1..19: note = signal peptide heavy chain
MNFGFSLIFLVLVLKGVQC

SEQ ID NO: 30 - protein, 455 aa, synthetic construct
  REGION 1..455: note = constant region heavy chain
ESQSFPNVFPLVSCESPLSDKNLVAMGCLARDFLPSTISFTWNYQNNTEVIQGIRTFPTLRTGGKYLATSQVLLSPKSILEGSDEYLVCKIHYGGKNRDLHVPIPAVAEMNPNVNVFVPPRDGFSGPAPRKSKLICEATNFTPKPITVSWLKDGKLVESGFTTDPVTIENKGSTPQTYKVISTLTISEIDWLNLNVYTCRVDHRGLTFLKNVSSTCAASPSTDILTFTIPPSFADIFLSKSANLTCLVSNLATYETLNISWASQSGEPLETKIKIMESHPNGTFSAKGVASVCVEDWNNRKEFVCTVTHRDLPSPQKKFISKPNEVHKHPPAVYLLPPAREQLNLRESATVTCLVKGFSPADISVQWLQRGQLLPQEKYVTSAPMPEPGAPGFYFTHSILTVTEEEWNSGETYTCVVGHEALPHLVTERTVDKSTGKPTLYNVSLIMSDTGGTCY

SEQ ID NO: 31 - DNA, 57 bp, synthetic construct
  misc_feature 1..57: note = signal peptide heavy chain
  source 1..57: mol_type = other DNA
atgaacttcgggttcagcttgattttccttgtccttgttttaaaaggtgtccagtgt

SEQ ID NO: 32 - DNA, 1365 bp, synthetic construct
  misc_feature 1..1365: note = constant region heavy chain
  source 1..1365: mol_type = other DNA
gagagtcagtccttcccaaatgtcttccccctcgtctcctgcgagagccccctgtctgataagaatctggtggccatgggctgcctggcccgggacttcctgcccagcaccatttccttcacctggaactaccagaacaacactgaagtcatccagggtatcagaaccttcccaacactgaggacagggggcaagtacctagccacctcgcaggtgttgctgtctcccaagagcatccttgaaggttcagatgaatacctggtatgcaaaatccactacggaggcaaaaacagagatctgcatgtgcccattccagctgtcgcagagatgaaccccaatgtaaatgtgttcgtcccaccacgggatggcttctctggccctgcaccacgcaagtctaaactcatctgcgaggccacgaacttcactccaaaaccgatcacagtatcctggctaaaggatgggaagctcgtggaatctggcttcaccacagatccggtgaccatcgagaacaaaggatccacaccccaaacctacaaggtcataagcacacttaccatctctgaaatcgactggctgaacctgaatgtgtacacctgccgtgtggatcacaggggtctcaccttcttgaagaacgtgtcctccacatgtgctgccagtccctccacagacatcctaaccttcaccatccccccctcctttgccgacatcttcctcagcaagtccgctaacctgacctgtctggtctcaaacctggcaacctatgaaaccctgaatatctcctgggcttctcaaagtggtgaaccactggaaaccaaaattaaaatcatggaaagccatcccaatggcaccttcagtgctaagggtgtggctagtgtttgtgtggaagactggaataacaggaaggaatttgtgtgtactgtgactcacagggatctgccttcaccacagaagaaattcatctcaaaacccaatgaggtgcacaaacatccacctgctgtgtacctgctgccaccagctcgtgagcaactgaacctgagggagtcagccacagtcacctgcctggtgaagggcttctctcctgcagacatcagtgtgcagtggcttcagagagggcaactcttgccccaagagaagtatgtgaccagtgccccgatgccagagcctggggccccaggcttctactttacccacagcatcctgactgtgacagaggaggaatggaactccggagagacctatacctgtgttgtaggccacgaggccctgccacacctggtgaccgagaggaccgtggacaagtccactggtaaacccacactgtacaatgtctccctgatcatgtctgacacaggcggcacctgctat

SEQ ID NO: 33 - protein, 19 aa, synthetic construct
  REGION 1..19: note = signal peptide light chain
MSPAQFLFLLVLWIRETNG

SEQ ID NO: 34 - protein, 107 aa, synthetic construct; identical to SEQ ID NO: 26
  REGION 1..107: note = constant region light chain

SEQ ID NO: 35 - DNA, 57 bp, synthetic construct
  misc_feature 1..57: note = signal peptide light chain
  source 1..57: mol_type = other DNA
atgagtcctgcccagttcctgtttctgttagtgctctggattcgggaaaccaacggt

SEQ ID NO: 36 - DNA, 321 bp, synthetic construct; identical to SEQ ID NO: 28
  misc_feature 1..321: note = constant region light chain
  source 1..321: mol_type = other DNA

(19) United States
(12) Patent Application Publication
(10) Pub. No.: US 2024/0352099 A1
(43) Pub. Date: Oct. 24, 2024

(54) ANTIBODIES AGAINST CANDIDA ALBICANS PROTEINS AND THEIR THERAPEUTIC AND PROPHYLACTIC USE FOR TREATING AND PREVENTING INVASIVE FUNGAL INFECTIONS

(71) Applicant: Julius-Maximilians-Universität Würzburg, Würzburg (DE)

(72) Inventors: Niklas Beyersdorf, Würzburg (DE); Daniela Langenhorst, Eisingen (DE); Peter F. Zipfel, Jena (DE); Prasad Dasari, Ulm (DE)

(21) Appl. No.: 18/685,658

(22) PCT Filed: Aug. 23, 2022
(86) PCT No.: PCT/EP2022/073476

§ 371 Date: Feb. 22, 2024

(30) Foreign Application Priority Data
Aug. 24, 2021 (EP) 21192859.3

(51) Int. Cl. C07K 16/14; A61K 39/00; A61P 31/10; A61P 37/04

(52) U.S. Cl. CPC C07K 16/14; A61P 31/10; A61P 37/04; A61K 2039/505

(57) ABSTRACT

Invasive fungal disease (IFD) constitutes an increasing health concern due to growing numbers of patients at risk for opportunistic fungal infections. Among the fungi capable of inducing opportunistic infections the yeast Candida albicans is clinically the most important. Immune evasion proteins like the pH-regulated antigen 1 (Pra1) and the translation elongation factor 1 (Tef1), which are expressed on the fungal surface and are also secreted, are major drivers of pathogenicity. Therefore, novel monoclonal antibodies (mAb) binding these proteins have been developed. In an in vivo mouse model of high-dose septic C. albicans infection, therapeutic application of mAb against Pra1 reduced clinical symptoms of the disease. Prophylactically, mAb against Tef1 protected mice from clinical disease and prolonged survival. The mABs of the present invention may also be efficacious in patients at risk or with already established IFD.

### FIELD OF THE INVENTION

The present invention is concerned with antibodies versus Candida albicans proteins and their use in infective therapy, immunotherapy, and the treatment or prevention of sepsis, in particular caused by a Candida infection, and specifically a Candida albicans infection.

### BACKGROUND

It is estimated that each year about 12% of the German population develop a fungal disease. Among the fungi capable of inducing opportunistic infections, the yeast Candida albicans is the most important pathogen. While the majority of these infections is only superficial, the incidence of invasive and, thus, life-threatening fungal infections increases continuously. Invasive candidiasis, which includes bloodstream infections (candidaemia), is the most frequently encountered invasive fungal disease (IFD). Furthermore, azole-resistant Candida strains are on the rise, causing an increase in infections.

IFD is becoming more important as more and more patients are at risk of acquiring an IFD due to the increasing standard of health care. It is patients with an organ transplant, after bone marrow transplantation, following extended surgery, patients on immunosuppressants, with HIV, or preterm births that are most at risk of developing an IFD. Another increasingly affected cohort of patients are those suffering from COPD (Chronic Obstructive Pulmonary Disease). Clinical care for patients with IFD is very challenging, as is reflected by mortality rates between 40 and 90% depending on the precise patient cohort and where the infection is localised.

The number of available antifungal drugs is very limited, particularly when considering differences in the mode of action. Most antifungal drugs interfere with biosynthesis or integrity of ergosterol, the major sterol in the fungal cell membrane. Others cause disruption of the fungal cell wall. Based on their mechanism of action, the major agents can be grouped into five classes: polyenes; azoles; allylamines; echinocandins; and other agents, including griseofulvin and flucytosine. The four main classes of antifungal drugs are the polyenes, azoles, allylamines and echinocandins, such as caspofungin. The currently available substances either target the cell wall, the endoplasmic reticulum or the DNA synthesis of the fungus. Patients at risk of developing IFD routinely receive one class of antifungal drug as prophylaxis. If patients then develop a ‘breakthrough’ infection under prophylactic medication, clinicians are routinely left with only one or two further classes of antifungal drugs to choose from for the treatment of such a breakthrough infection. Therefore, drug resistance is a common complication and one of the reasons for the high mortality rates due to IFD.

In the past, efforts have been made to develop monoclonal antibodies for use in the treatment of opportunistic fungal infections. Mycograb (Efungumab) targeting fungal heat shock protein 90 (HSP90) was developed by NeuTec Pharma to treat IFD (see Matthews et al., 2003, Antimicrobial agents and chemotherapy 47:2208-2216). In an in vivo study on sublethal IFD Mycograb showed efficacy and the combination of Mycograb with the polyene antifungal amphothericin B (AMB) enhanced fungal clearance compared to treatment with either drug alone. In this study, the effect of Mycograb was, however, not controlled for by injection of an antibody (fragment) of irrelevant specificity. Therefore, it is not clear whether Mycograb actually elicited a specific inhibitory effect on fungal growth. This concern was further corroborated by the finding that the potentiation of the antifungal activity of AMB by Mycograb in vitro was unspecific as Mycograb could be substituted for by immunoglobulin of irrelevant specificity or even albumin (see Richie et al, 2012, Antimicrobial agents and chemotherapy 56:3963-3964). Due to problems referring to the quality of the drug, i.e. its propensity to fold or form aggregates, immunogenicity and induction of an unexplained cytokine release syndrome, Efungumab was not granted market authorization. A modified follow-up version of Mycograb (C28Y) with increased molecular stability lacked efficacy in vivo

Recently, human monoclonal antibodies with reactivity against the cell wall of C. albicans, specifically the cell wall protein Hyr1, have been isolated from the peripheral blood of convalescent patients with superficial candidiasis (see Rudkin et al., 2018, Nat Commun 9:5288 and international patent publication WO2016142660). These mAb recognize polysaccharide epitopes expressed by many different Candida subspecies. In an in vivo infection model pre-coating of C. albicans strain SC5314 with antifungal cell wall mAb prior to intravenous injection reduced the fungal burden in the kidneys of mice three days after inoculation.

β-glucan is a major cell wall component of fungi recognized by the pathogen recognition receptor Dectin-1 and the target of echinocandin antifungals. To further exploit β-glucan as a therapeutic target, mAb were generated in mice against β-(1-3)-D-glucan (see Matveev et al., 2019, PloS one 14: e0215535). As expected, these antibodies bind to the surface of a very broad spectrum of fungi including Candida and Aspergillus subspecies. Antifungal activity of these antibodies was demonstrated against A. fumigatus and C. albicans in vitro and in a model of systemic C. albicans infection in vivo.

Apart from the human antifungal mAb and the mouse anti-β-glucan mAb, which have both been evaluated in in vivo infection models in mice, mAb with reactivity for the fungal cell adhesin Als3 have been generated and shown to inhibit the growth of C. albicans, A. fumigatus and other fungi in vitro (see Moragues et al, 2003. Infect Immun 71:5273-5279, and Brena et al., 2007, Infect Immun 75:3680-3682). There are currently, however, no data available on the in vivo efficacy of these antibodies in IFD.

Therefore, despite the above-mentioned previous efforts, there are currently no monoclonal antibodies (mAb) approved for the treatment of opportunistic fungal infections.

As presented above, the therapeutics available to treat or prevent IFD are currently very limited. Often IFD manifests itself in patients despite prophylactic treatment with one of the antifungal drugs available. In such instances, applying an antifungal drug of another class may also not be capable of stopping fungal growth in patients.

Therefore, novel drugs for treating and/or preventing IFD are urgently needed.

### SUMMARY OF THE INVENTION

Over the last years, it has been increasingly recognised that immune evasion proteins strongly contribute to fungal pathogenicity. For C. albicans, the switch from the harmless yeast to the pathogenic hyphal form is accompanied by strongly increased expression of immune evasion proteins. So far, therapeutic targeting of immune evasion proteins with antibodies has not been achieved.

This technical problem has now been surprisingly solved by generating antibodies against the fungal immune evasion proteins Pra1 and Tef1, and by confirming that these immune evasion proteins can be efficiently targeted for the protection from systemic C. albicans infections in vivo. Homologue proteins to Pra1 and Tef1 exist in other Candida species, which makes it plausible that the antibodies of the invention will also be able to inhibit Pra1 or Tef1 homologues in other Candida species

Therefore, the present invention provides antibodies against the Candida albicans proteins Pra1 and Tef1, which are efficacious in treating or preventing a Candida albicans infection in a subject.

The pH regulated antigen 1 (Pra1) is a fungal immune evasion protein of C. albicans. Pra1 expression correlates with pathogenicity as it is highly expressed on hyphae, but not C. albicans yeast cells. Pra1 interferes with complement activation and, thus, fungal opsonization and inflammation by recruiting the soluble complement regulator Factor H and Plasminogen to the fungal surface and by directly binding to C3 and its cleavage products. Moreover, Pra1 also inhibits interferon (IFN)γ secretion by T helper (Th) 1 cells involved in fungal clearance. As a moonlighting protein, Pra1 supplies C. albicans with Zn²⁺ and is involved in biofilm formation. In addition, Pra1 has been described as the major ligand for complement receptor 3, i.e. CD11b/CD18 integrin. A Pra1-deficient C. albicans strain, thus, grew more aggressively than wild-type C. albicans in wild-type mice as did wild-type C. albicans in CD11b-deficient mice.

Similar to Pra1, Translation elongation factor-1 alpha (Tef1) of C. albicans is also more strongly expressed on the surface of hyphae than on C. albicans yeast cells and is also secreted. It binds Factor H, Plasminogen and C3 to inhibit complement activity. In addition, Tef1 directly binds to human B cells via complement receptor 2 (CD21) and induces a regulatory phenotype in the B cells marked by secretion of anti-inflammatory IL-10. Tef1, thus, mediates immune evasion by complement inhibition and induction of regulatory B cells.

Immune evasion proteins like the pH-regulated antigen 1 (Pra1) and the translation elongation factor 1 (Tef1) of C. albicans, which are expressed on the fungal surface and are also secreted, are major drivers of pathogenicity. Therefore, it has been found that these proteins are promising targets for immunotherapy to prevent and/or treat invasive fungal infections caused by pathogens expressing these proteins.

Therefore, novel monoclonal antibodies (mAb) binding these proteins have been provided by this invention. Using an in vivo model of high-dose septic C. albicans infection, it was observed that therapeutic application of mAb against Pra1 reduced clinical symptoms of the disease. Prophylactically, mAb against Tef1 protected mice from clinical disease and prolonged survival. Together, the data presented in this application indicates that targeting immune evasion proteins of opportunistic fungi with mAb may also be efficacious in patients at risk or with already established IFD.

### BRIEF DESCRIPTION OF THE DRAWINGS

FIG. 1 shows the result of a Western blot testing the binding capability of mAB 8C3 against Pra1 (FIG. 1A), of mAB 1E12 against Pra1 (FIG. 1B), and of mAb 5E1 against Tef1 (FIG. 1C).

FIG. 2 shows the results of light microscopy (40× magnification) of gram-stained cultured C. albicans cells. The hyphae formation over time is demonstrated.

FIG. 3 shows the results of a flow cytometric analysis at different time points after induction of hyphal growth in C. albicans. It is shown that the respective antibodies bound their antigens only after induction of hyphal growth.

FIG. 4 shows the results of confocal microscopy to visualise binding of antibody 5E1 to Tef1 in C. albicans hyphae, but not C. albicans cells.

FIG. 5 shows therapeutic efficacy of the respective antibodies compared to a control in mice after systemic infection with C. albicans. Clinical score (see also Table 2) was charted versus days after infection. The respective antibodies or controls were injected on day 1 after infection in mice (two-way ANOVA ‘treatment’: p<0.0001; Tukey post-hoc test: *p<0.05, **p<0.01).

FIG. 6 shows therapeutic efficacy of the antifungal drug Caspofungin compared to a control in mice after systemic infection with C. albicans (two-way ANOVA ‘treatment’: p<0.0001; Tukey post-hoc test: ***p<0.001, ****p<0.0001).

FIG. 7 shows survival rate of mice after systemic infection with C. albicans and treatment with the respective antibodies and controls (log-rank test *p<0.05).

FIG. 8 shows survival rate of mice after systemic infection with C. albicans and treatment with different amounts of the antifungal drug Caspofungin (log-rank test **p<0.01).

FIG. 9 shows the fungal burden in kidneys of sacrificed mice treated with antibodies or caspofungin.

FIG. 10 shows results of prophylactic treatment with respective antibodies of mice one day before systemic infection with C. albicans (two-way ANOVA ‘treatment’: p<0.0001; Tukey post-hoc test: *p<0.05, **p<0.01, ***p<0.001, ****p<0.0001).

FIG. 11 shows survival rate of mice after systemic infection with C. albicans and prophylactic treatment with the respective antibodies one day before systemic infection with C. albicans (log-rank test: *p<0.05).

FIG. 12 shows the fungal burden in kidneys of sacrificed mice treated with the respective antibodies.

FIG. 13 shows a scheme of how mAb 5E1 reverses the inhibitory effect of Tef1 on complement component C3.

FIG. 14 summarizes data on the blockade of the Tef1 effect on C3 attachment to the surface of C. albicans yeast cells.

FIG. 15 shows that mAb 5E1 increases phagocytosis of C. albicans yeast cells by mouse bone marrow cells.

### DESCRIPTION OF THE INVENTION

The present invention is concerned with an antibody directed against Pra1 of Candida albicans, or an antibody fragment thereof, comprising a complementarity-determining region 1 (CDR1), which comprises an amino acid sequence as defined by SEQ ID NO:1, a complementarity-determining region 2 (CDR2), which comprises an amino acid sequence as defined by SEQ ID NO:2, a complementarity-determining region 3 (CDR3), which comprises an amino acid sequence as defined by SEQ ID NO:3, a complementarity-determining region 4 (CDR4) as defined by SEQ ID NO:4, a complementarity-determining region 5 (CDR5), which comprises an amino acid sequence as defined by SEQ ID NO:5, and a complementarity-determining region 6 (CDR6), which comprises an amino acid sequence as defined by SEQ ID NO:6, wherein any one of the CDR sequences can be altered by substitution, deletion, or insertion of 1 or 2 amino acids, provided that the resulting antibody substantially maintains the functionality of the antibody comprising the unaltered CDRs as defined by SEQ ID NOs: 1-6.

In one embodiment, the invention is concerned with an antibody, or a fragment thereof, comprising the CDRs as defined by SEQ ID NOs: 1-6.

In a further embodiment, the invention is concerned with an antibody, or a fragment thereof, comprising a variable heavy chain as defined by SEQ ID NO: 13, and a variable light chain as defined by SEQ ID NO: 14.

In a further embodiment, the invention is concerned with an antibody, or a fragment thereof, comprising a variable heavy chain, which has at least 90% identity with the sequence as defined by SEQ ID NO: 13, and a variable light chain, which has at least 90% identity with the sequence as defined by SEQ ID NO: 14.

In a further embodiment, the invention is concerned with an antibody, or a fragment thereof, comprising the CDRs as defined by SEQ ID NOs: 1-3 included in a variable heavy chain, which has at least 90% identity with the sequence as defined by SEQ ID NO: 13, and the CDRs as defined by SEQ ID NOs: 4-6 included a variable light chain, which has at least 90% identity with the sequence as defined by SEQ ID NO: 14.

One exemplary antibody of the present invention comprising the CDRs as defined by SEQ ID NOs. 1-6 is the monoclonal antibody 8C3 directed against Pra1 of Candida albicans, produced by the hybridoma cell line with the accession number DSM ACC 3369.

The present invention is also concerned with the hybridoma cell line deposited with accession number DSM ACC 3369, which is capable of producing the monoclonal antibody 8C3 directed against Pra1 of Candida albicans (8C3).

The present invention is also concerned with a pharmaceutical composition comprising the anti-Pra1 antibody of the invention, or a fragment thereof, and optionally pharmaceutically acceptable excipients and/or carriers.

The anti-Pra1 antibody of the present invention or a fragment thereof or the pharmaceutical composition comprising the anti-Pra1 antibody of the invention or the fragment thereof, can be used therapeutically in a method of treating an invasive fungal disease in a subject. The invasive fungal disease is preferably caused by the yeast Candida, and specifically by the yeast Candida albicans. The antibody of the present invention is administered systemically, preferably by injection, at least once.

The present invention is also concerned with an antibody directed against Tef1 of Candida albicans, or an antibody fragment thereof, comprising a complementarity-determining region 1 (CDR1), which comprises an amino acid sequence as defined by SEQ ID NO: 7, a complementarity-determining region 2 (CDR2), which comprises an amino acid sequence as defined by SEQ ID NO:8, a complementarity-determining region 3 (CDR3), which comprises an amino acid sequence as defined by SEQ ID NO:9, a complementarity-determining region 4 (CDR4), which comprises an amino acid sequence as defined by SEQ ID NO: 10, a complementarity-determining region 5 (CDR5), which comprises an amino acid sequence as defined by SEQ ID NO:11, and a complementarity-determining region 6 (CDR6), which comprises an amino acid sequence as defined by SEQ ID NO: 12, wherein any one of the CDR sequences can be altered by substitution, deletion, or insertion of 1, or 2 amino acids, provided that the resulting antibody substantially maintains the functionality of the antibody comprising the unaltered CDRs as defined by SEQ ID Nos: 1-6.

In one embodiment, the invention is concerned with an antibody, or a fragment thereof, comprising the CDRs as defined by SEQ ID Nos: 7-12.

In a further embodiment, the invention is concerned with an antibody, or a fragment thereof, comprising a variable heavy chain as defined by SEQ ID NO: 15, and a variable light chain as defined by SEQ ID NO: 16.

In a further embodiment, the invention is concerned with an antibody, or a fragment thereof, comprising a variable heavy chain, which has at least 90% identity with the sequence as defined by SEQ ID NO: 15, and a variable light chain, which has at least 90% identity with the sequence as defined by SEQ ID NO: 16.

In a further embodiment, the invention is concerned with an antibody, or a fragment thereof, comprising the CDRs as defined by SEQ ID NOs: 7-9 included in a variable heavy chain, which has at least 90% identity with the sequence as defined by SEQ ID NO: 15, and the CDRs as defined by SEQ ID NOs: 10-12 included a variable light chain, which has at least 90% identity with the sequence as defined by SEQ ID NO: 16.

One exemplary antibody of the present invention comprising the CDRs as defined by SEQ ID Nos: 7-12 is the monoclonal antibody 5E1 directed against Tef1 of Candida albicans, produced by the hybridoma cell line with the accession number DSM ACC 3368.

The present invention is also concerned with the hybridoma cell line deposited with accession number DSM ACC 3368, which is capable of producing the monoclonal antibody 5E1 directed against Tef1 of Candida albicans (5E1).

The present invention is also concerned with a pharmaceutical composition comprising the anti-Tef1 antibody of the invention, or a fragment thereof, and optionally pharmaceutically acceptable excipients and/or carriers.

“Substantially maintaining the functionality of an antibody” in the context of this invention means that the altered antibody is a functional variant and displays substantially the same binding affinity, avidity, and/or specificity compared to the unaltered antibody.

“Substantially the same” means that the altered antibody exhibits a binding affinity, avidity, and/or specificity, which is at least 80% of the respective affinity, avidity, and specificity of the unaltered antibody.

A “functional variant” of an antibody of the present invention is any antibody or fragment thereof that has an affinity for the Candida albicans protein of the present invention that is at least 80%, more preferably at least 90% or at least 95% or even 99% or more than the specific antibody of the present invention, such as the antibody comprising the CDRs as defined. The affinity of a functional variant and of the specific antibody can be measured as is known in the art and the results can be compared as is known to the skilled person and by well-known assays, for example by surface plasmon resonance (SPR), or by other protein-protein interaction monitoring assays.

The amino acid substitution is preferably a conservative substitution. A “conservative substitution” refers to the substitution of one amino acid by another, wherein the replacement results in a silent alteration. This means that one or more amino acid residues within the CDR sequence of the present invention can be substituted by another amino acid of a similar polarity, which acts as a functional equivalent. Substitutes for an amino acid within the sequence may be selected from other members of the class to which the amino acid belongs (i.e. a conservative substitution). For example, one polar amino acid can be substituted by another polar amino acid; one positively or negatively charged amino acid, respectively, can be substituted by another positively or negatively charged amino acid, respectively, et cetera. Classes of amino acids are for example, nonpolar (hydrophobic) amino acids including alanine, leucine, isoleucine, valine, proline, phenylalanine, tryptophan and methionine; polar neutral amino acids including glycine, serine, threonine, cysteine, tyrosine, asparagine, and glutamine; positively charged (basic) amino acids including arginine, lysine and histidine; negatively charged (acidic) amino acids including aspartic acid and glutamic acid.

The terms “identical” or “percent identity”, in the context of two or more nucleic acids or polypeptide sequences, refer to two or more sequences or subsequences that are the same or have a specified percentage of amino acid residues or nucleotides that are the same (i.e., about 90%, 91%, 92%, 93%, 94%, 95%, 96%, 97%, 98%, 99%, or higher identity over a specified region, when compared and aligned for maximum correspondence over a comparison window or designated region) as measured using a BLAST or BLAST 2.0 sequence comparison algorithms with default parameters known to the skilled person, or by manual alignment and visual inspection (see, e.g., NCBI web site or the like). Such sequences are then said to be substantially identical. The definition also includes sequences that have deletions and/or additions, as well as those that have substitutions. The preferred algorithms can account for gaps and the like as is known in the art.

The term “polypeptide”, as used herein, generally refers to a polymer of at least three amino acids and is intended to include peptides and proteins, such as variable light chains and variable heavy chains of antibodies. The skilled person will appreciate, however, that the term polypeptide is intended to be sufficiently general as to encompass not only polypeptides having the complete sequence recited herein (or in a reference or database specifically mentioned herein), but also to encompass polypeptides, such as antibodies, that represent functional fragments (i.e., fragments retaining at least one activity) of such complete polypeptides. Moreover, the skilled person understands that protein sequences generally tolerate some substitution without destroying activity.

The term “antibody” in the meaning of the present invention typically refers to full-length antibodies and to antibody fragments of the aforementioned antibodies as well as variants as defined below. Antibodies that do not contain all the domains or regions of a full-length antibody, are fragments of antibodies within the meaning of the present invention. Thus, the term “antibody” shall encompass any type of antibody, fragments and variants thereof, and mixtures of antibodies, fragments, and/or variants. The antibody of the present invention can be used as whole antibody, or as a fragment thereof, wherein the fragment comprises the CDRs as defined for the whole antibody. The antibody of the invention can be a monoclonal antibody.

The antibody of the present invention can be a human antibody, a murine antibody, a chimeric antibody, or a humanized antibody. In a preferred embodiment, the antibody of the present invention is a humanized antibody.

A pharmaceutically acceptable excipient may be a compound or a combination of compounds entering into a pharmaceutical composition which does not provoke secondary reactions and which allows, for example, facilitation of the administration of the antibody of the present invention, an increase in its lifespan and/or in its efficacy in the body or an increase in its solubility in solution. These pharmaceutically acceptable vehicles are well known and will be adapted by the person skilled in the art as a function of the mode of administration of the antibody of the present invention.

The anti-Tef1 antibody of the present invention or a fragment thereof or the pharmaceutical composition comprising the anti-Tef1 antibody of the invention or the fragment thereof, can be used therapeutically in a method of preventing, suppressing, or delaying the emergence of an invasive fungal disease in a subject. The invasive fungal disease is preferably caused by the yeast Candida, and specifically by the yeast Candida albicans. The antibody of the present invention is administered systemically, prior to manifestation of an IFD, preferably by injection, at least once.

The subjects to be treated with the antibodies of the invention suffer from an IFD or are at risk of acquiring an IFD. These subjects can be patients who have received an organ transplant, or a bone marrow transplantation, patients in recuperation after extended surgery, patients on immunosuppressants, patients diagnosed with HIV, or patients suffering from COPD (Chronic Obstructive Pulmonary Disease).

In another embodiment of the invention, the subject at risk of acquiring an IFD is prophylactically treated with the anti-Tef1 antibody of the invention or a fragment thereof or the pharmaceutical composition comprising the anti-Tef1 antibody of the invention or a fragment thereof, to prevent, suppress or delay the emergence of an IFD, followed by a therapeutic treatment with the anti-Pra1 antibody of the present invention or a fragment thereof or the pharmaceutical composition comprising the anti-Pra1 antibody of the invention or a fragment thereof, if the subject subsequently acquires an IFD.

In another embodiment of the invention, the treatment or the preventing of an IFD with the antibodies of the present invention can be combined with other antifungal drugs, such as caspofungin.

### EXAMPLES

The invention will now be further described with reference to the following non-limiting examples.

### Example 1—Generation of mAb Targeting Immune Evasion Proteins

All mAb were generated using the hybridoma technique. Specifically, female BALB/c mice were immunized twice subcutaneously with a four-week interval between injections with 10 μg of purified fungal protein together with the adjuvant TiterMax®. After another three weeks, the mice received an intravenous injection of fungal protein without adjuvant and were sacrificed after another three days to obtain splenocytes for mAb isolation.

### Example 2—Antifungal mAb Recognise Linear Protein Epitopes

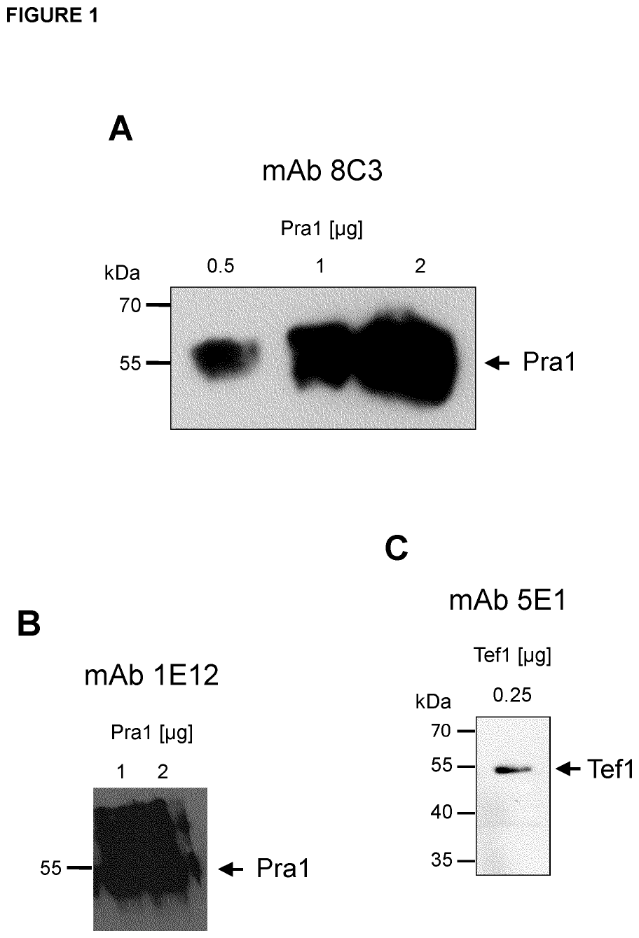

To test whether the antifungal mAb generated by the inventors (Table 1) recognise linear or conformational epitopes of the antigens they were raised against, their binding to recombinant Pra1 (FIGS. 1A,B) and Tef1 (FIG. 1C) of C. albicans was tested in Western Blot. MAb 8C3 (FIG. 1A) and 1E12 (FIG. 1B) as well as mAb 5E1 (FIG. 1C) bound recombinant Pra1 and Tef1, respectively, on Western Blot. Therefore, all antibodies recognised linear epitopes of Pra1 and Tef1, respectively.

TABLE 1
Overview over the monoclonal antibodies
generated for this invention.

| | Immune | | |
|---|---|---|---|
| | evasion | | |
| Fungus | protein | Clone | Isotype |
| C. albicans | Pra1 | 8C3 | IgG₁, κ |
| | | 1E12 | IgG₁, κ |
| | Tef1 | 5E1 | IgM, κ |

### Example 3—C. albicans Hyphae are the Prime Targets of Antifungal mAb

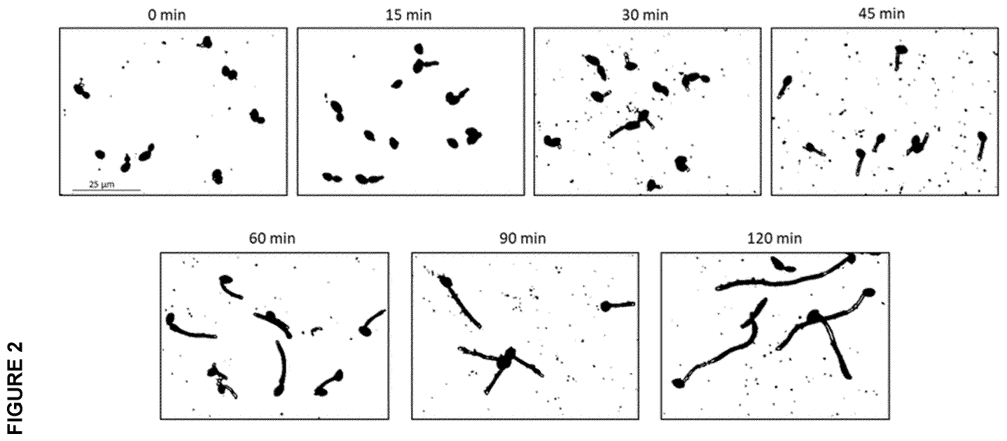

C. albicans pathogenicity is positively correlated with the change from yeast to hyphae. To study binding of antifungal mAb to different morphotypes, hyphae formation was induced by addition of 20% fetal calf serum and culturing C. albicans strain SC5314 cells for up to 120 min at 37° C. (FIG. 2). At different time points, a gram staining of the cultured C. albicans cells was done and hyphae formation was analysed by light microscopy (FIG. 2, 40× magnification). With this protocol hyphae formation was already visible after 15 min and hyphal growth steadily increased until 120 min (FIG. 2).

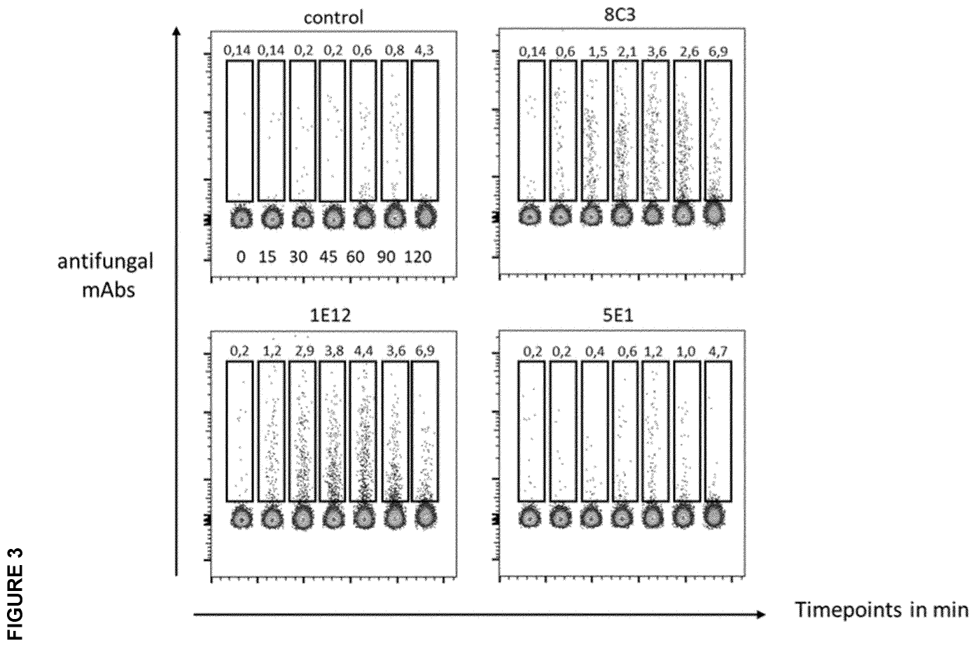

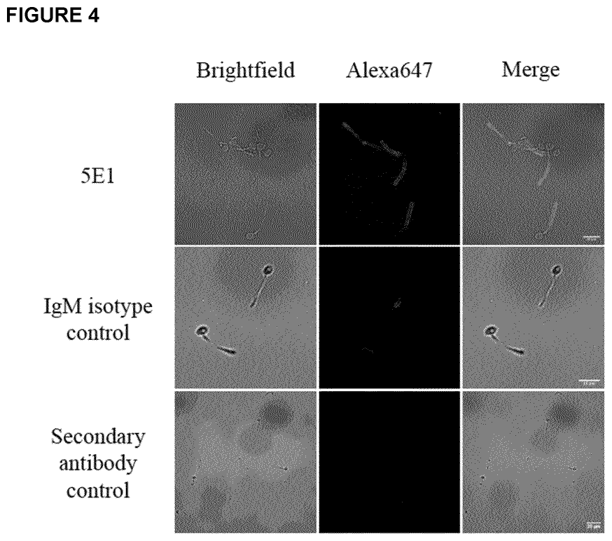

Staining of C. albicans with mAb 8C3, 1E12, 5E1 or an IgG₁ mAb of irrelevant specificity at different time points after induction of hyphal growth followed by flow cytometric analysis showed that the antifungal mAb only bound C. albicans after hyphae induction (FIG. 3). mAb were used at 10 μg/ml for these stainings and bound antibodies were detected with an Alexa Fluor 647-coupled goat anti-mouse IgG secondary antibody. Confocal microscopy was used to directly visualise binding of mAb 5E1 recognising Tef1 to C. albicans hyphae (FIG. 4). While the hyphae were brightly stained with 5E1 and Alexa Fluor 647-coupled goat anti-mouse IgG secondary antibody, this was not the case for C. albicans cells (FIG. 4, top row) or when using an IgM isotype control antibody of irrelevant specificity (FIG. 4, middle row). Secondary antibody alone also gave no specific signal (FIG. 4, bottom row).

Together, the data presented in this description indicates that the antifungal mAb specifically recognise C. albicans hyphae, but not yeast cells.

### Example 4—Mouse Model for IFD

To induce IFD, 3×10⁵ SC5314 C. albicans cells were injected into female BALB/c mice intravenously. After disease induction, the mice were scored twice daily and the animals were euthanised when they reached a humane endpoint or at the pre-scheduled end of the experiment 14 days after infection (Table 2).

TABLE 2
Scoring parameters.

| | Score 0 | Score 1 | Score 2 | Score 3 |
|---|---|---|---|---|
| 1.) Weight loss | <7% | >7%, <14% | >14%, <20% | ≥20% |
| 2.) Posture | normal | hunching | hunching | severe hunching |
| | | noted only | also when | impairs |
| | | at rest | active | movement |
| 3.) Activity | normal | mildly | moderately | severely |
| | | decreased | decreased | decreased but |
| | | | | still active |
| 4.) Fur texture | normal | mild ruffling | moderate | severe |
| | | | ruffling | ruffling/poor |
| | | | | grooming |

Humane endpoints were:
total score of 9 or higher
weight loss score 3
activity score 3

### Example 5—Therapeutic Application of mAb in Mouse Model

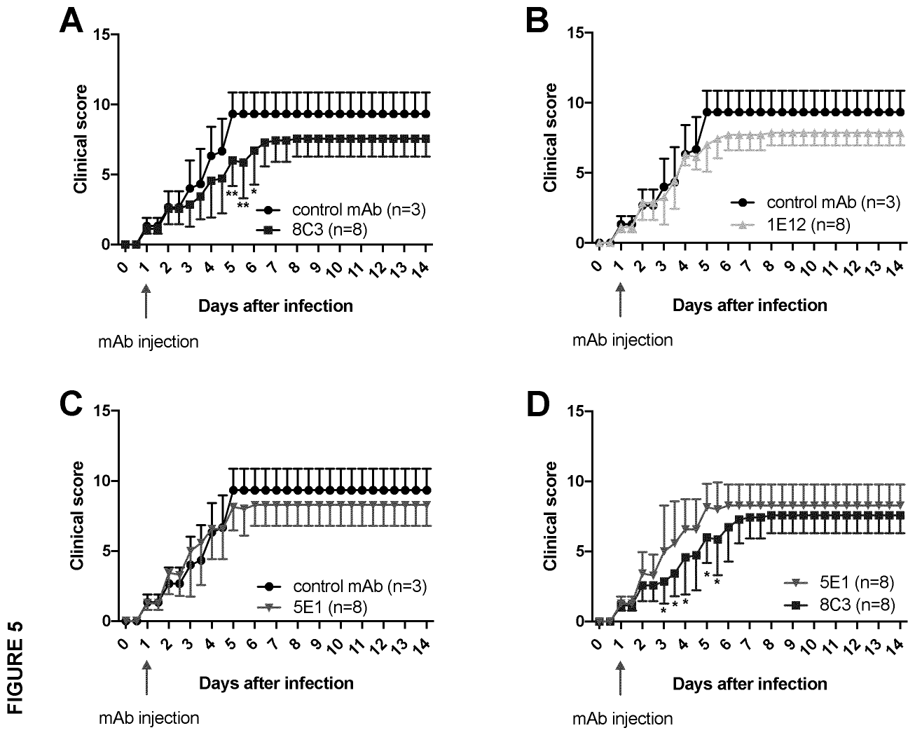

To test for therapeutic efficacy, 100 μg/mouse of the anti-Pra1 mAb 8C3 or 1E12 or the anti-Tef1 mAb 5E1 or mAb MOPC-21 (mouse IgG₁, κ; irrelevant specificity) were injected one day after systemic infection of mice with C. albicans (FIG. 5). mAb 8C3 significantly improved the clinical score compared to control mAb MOPC-21-injected mice (FIG. 5A), which was not the case for mAb 1E12 (FIG. 5B) and 5E1 (FIG. 5C). Comparing the impact of the different mAb on the clinical score showed that mice receiving mAb 8C3 did significantly better than animals receiving mAb 5E1 (FIG. 5D; n=8 for both groups).

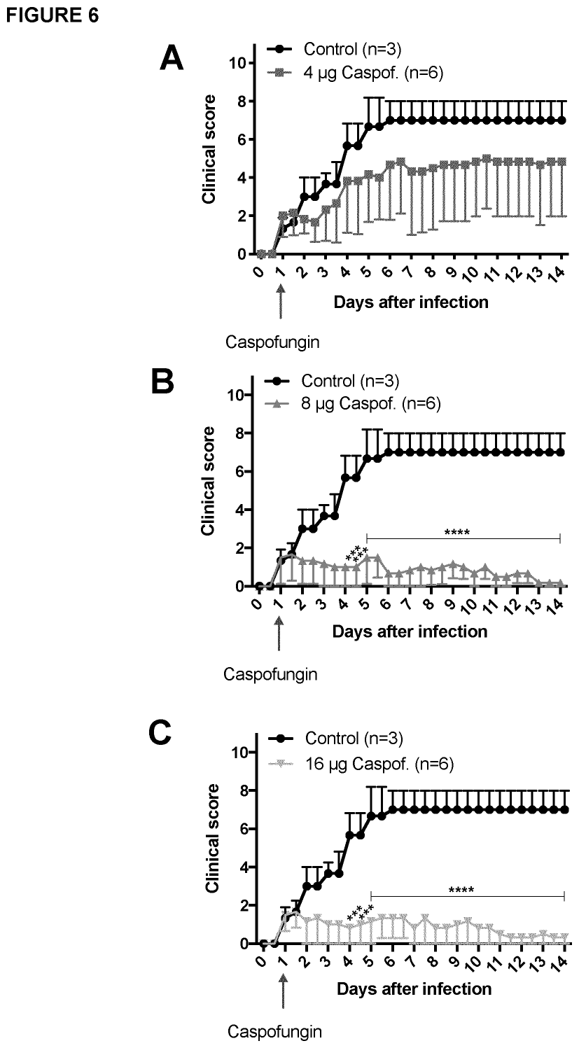

In a separate set of experiments, the therapeutic efficacy of the echinocandin antifungal drug caspofungin in the mouse model used in the present invention was tested. Here, a single injection of 4 μg (about 160 μg/kg BW) led to lower clinical scores compared to controls, but without reaching statistical significance. Dosages of 8 and 16 μg per mouse (about 320 and 640 μg/kg BW) significantly lowered clinical scores from day four after infection onwards (FIG. 6). For comparison, in humans caspofungin is applied for the treatment of IFD caused by C. albicans at about 1 mg/kg BW every 24 h.

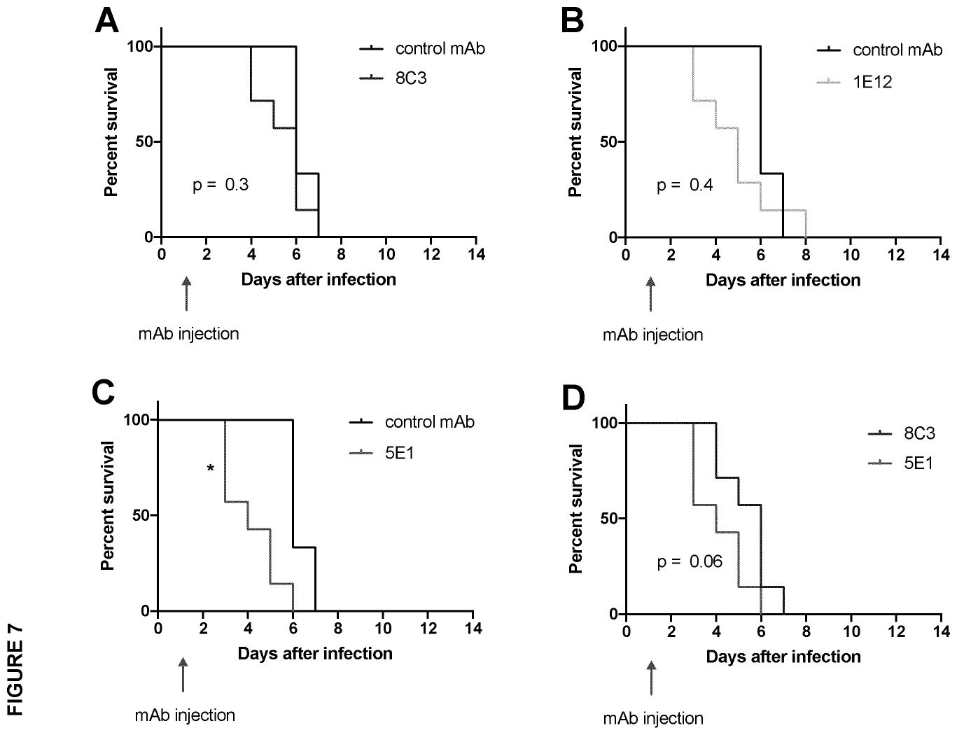

Regarding survival, there were no differences between C. albicans-infected mice receiving mAb 8C3 or 1E12 versus control mAb MOPC-21 (FIGS. 7A, B). However, mice receiving mAb 5E1 died on average two days earlier than MOPC-21-treated animals (FIG. 7C). The comparison of mAb 8C3 versus mAb 5E1-treated animals showed that there was a clear trend towards a better survival of 8C3 versus 5E1-treated animals (FIG. 7D).

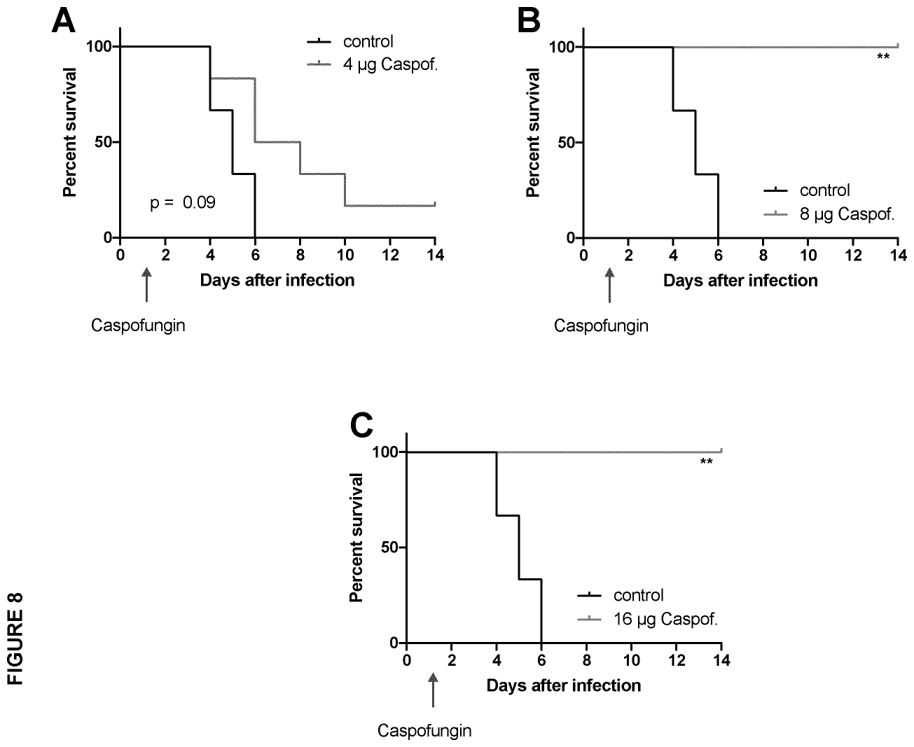

A single caspofungin injection of 8 or 16 μg per mouse fully prevented the mice from being euthanised for humane reasons (FIGS. 8B, C), while the positive effect of 4 μg caspofungin/mouse did not reach statistical significance (FIG. 8A).

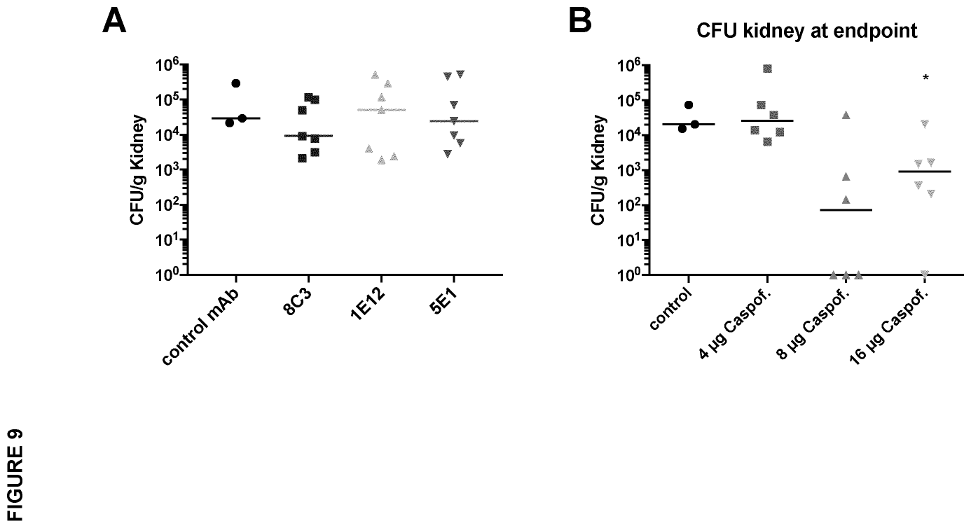

In post-mortem analyses of mice killed for humane reasons (FIGS. 7, 8) the fungal burden in the kidney (FIG. 9) was determined. By the time the mice reached the humane endpoints, there were no differences in fungal burden between the groups treated with antifungal mAb and control mAb (FIG. 9A). Mice receiving 8 or 16 μg caspofungin, however, had comparatively low fungal burdens at the end of the experiment, i.e. 14 days after infection (FIG. 9B).

The data presented herein thus shows that treatment of mice with a single injection of mAb 8C3 mitigates IFD caused by C. albicans.

### Example 6—Prophylactic Application of mAb in Mouse Model

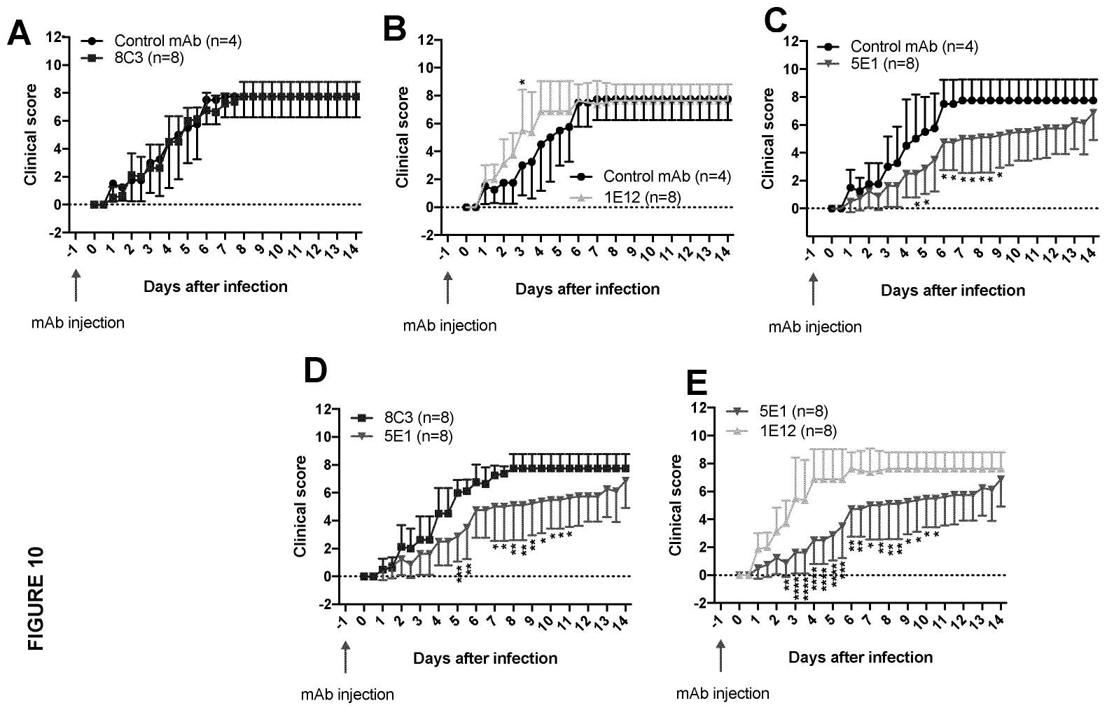

As many patients at risk of developing opportunistic fungal infections receive prophylactic treatment, 100 μg/mouse of the anti-Pra1 mAb 8C3 or 1E12 or the anti-Tef1 mAb 5E1 or mAb MOPC-21 (mouse IgG₁, κ; irrelevant specificity) were injected one day before systemic infection of mice with C. albicans to test for prophylactic activity of the antifungal mAb (FIG. 10). Regarding clinical responses, only mice treated with mAb 5E1 had a lower clinical score than controls (FIG. 10A-C). Comparing 5E1- to 8C3- (FIG. 10D) and 1E12-treated mice (FIG. 10E) showed that 5E1-treated mice did significantly better than 8C3- or 1E12-treated animals, respectively (n=8 for all groups).

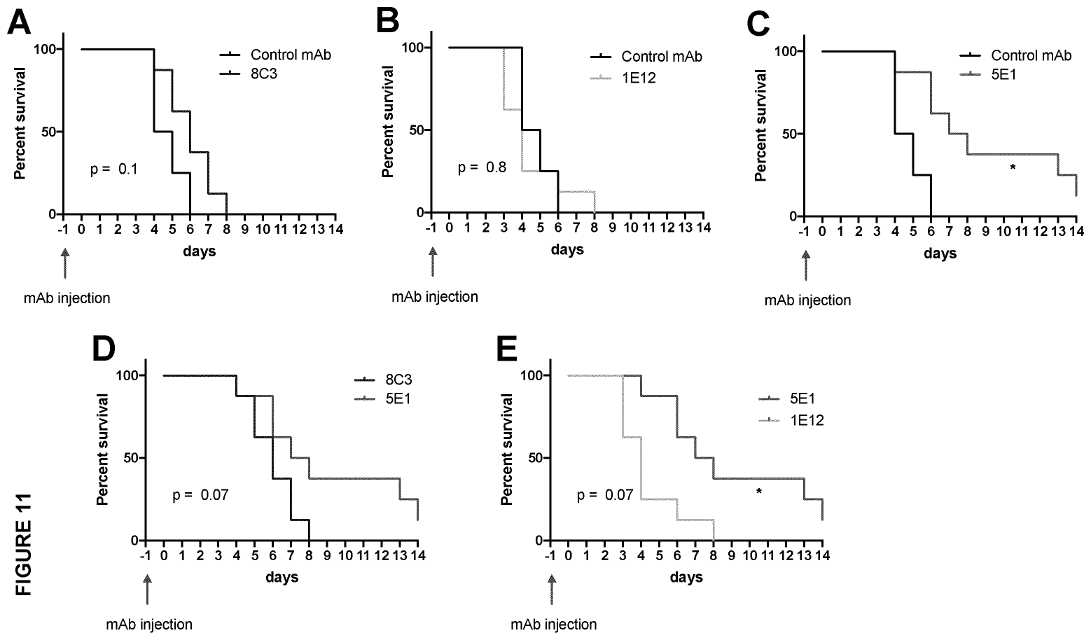

The lower clinical score of 5E1-treated animals translated into longer survival compared to control-mAb-treated mice (FIG. 11C), which was not the case for 8C3- (FIG. 11A) and 1E12-treated animals (FIG. 11B). 5E1-treated mice also had a survival advantage compared to 1E12-injected animals (FIG. 11E), while the comparison of 8C3- and 5E1-treated mice was not statistically significant (FIG. 11D).

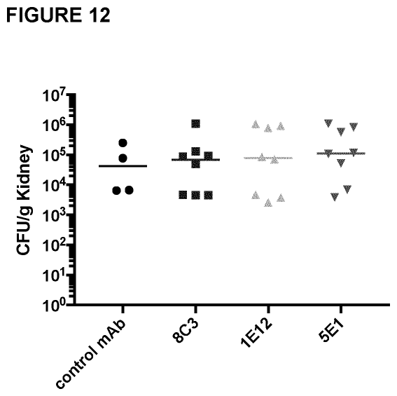

Again, fungal burden was high for all mice killed either for humane reasons or at the end of the two-week observation period (FIG. 12).

Taken together, prophylactic application of mAb 5E1 protected mice from C. albicans-induced IFD.

### Example 7—Sequencing of CDRs of Antibodies of the Invention

The nucleotide sequences and the amino acid sequences of antibodies of the present invention were identified.
Table 3 shows the regions of amino acid sequences defining the CDRs of the anti-Pra1 antibody produced by the hybridoma cell line 8C3 (Accession number: DSM ACC 3369).
CDR-H1, CDR-H2, and CDR-H3 correspond to CDR1, CDR2, and CDR3 respectively, and are located on the heavy variable chain of the antibody.
CDR-L1, CDR-L2, and CDR-L3 correspond to CDR4, CDR5, and CDR6 respectively, and are located on the light variable chain of the antibody.

TABLE 3
Regions of amino acid sequence defining the CDRs
of anti-Pra1 antibody 8C3:

| Region | Sequence |
|---|---|
| CDR-H1 | TYGMS (SEQ ID NO: 1) |
| CDR-H2 | TISSGGSYTYYPDSVKG (SEQ ID NO: 2) |
| CDR-H3 | QGLDDNYAEWYFDV (SEQ ID NO: 3) |
| CDR-L1 | RASQSIYKNLH (SEQ ID NO: 4) |
| CDR-L2 | YASDSIS (SEQ ID NO: 5) |
| CDR-L3 | LQGFSTPWT (SEQ ID NO: 6) |

The amino acid sequence of the variable heavy chain of the anti-Pra1 antibody produced by the hybridoma cell line 8C3 (Accession number: DSM ACC 3369) is defined by SEQ ID NO: 13:

EVQLVESGGDLVKPGGSLKLSCAASGFTFSTYGMSWVRQTPDKRLEWVA
TISSGGSYTYYPDSVKGRFTISRDNVKNTLYLQMSSLKSEDTAMYYCAR
QGLDDNYAEWYFDVWGAGTTVTVSS

The CDRs are bolded and are flanked by the framework regions (FR).

The amino acid sequence of the variable light chain of the anti-Pra1 antibody produced by the hybridoma cell line 8C3 (Accession number: DSM ACC 3369) is defined by SEQ ID NO: 14:

DILLTQSPATLSVTPGETVSLSCRASQSIYKNLHWYQQKSHRSPRLLIK
YASDSISGIPSRFTGSGSGTDYTLSINSVKPEDEGKYYCLQGFSTPWTF
GGGTKLEIK

The CDRs are bolded and are flanked by the framework regions.

The nucleotide sequence of the variable heavy chain of the anti-Pra1 antibody produced by the hybridoma cell line 8C3 (Accession number: DSM ACC 3369) is defined by SEQ ID NO: 17:

GAGGTCCAGCTGGTGGAATCTGGGGGAGACTTAGTGAAGCCTGGAGGGT
CCCTGAAACTCTCCTGTGCAGCCTCTGGATTCACTTTCAGTACCTATGG
CATGTCTTGGGTTCGCCAGACTCCAGACAAGAGGCTGGAGTGGGTCGCA
ACCATTAGTAGTGGTGGTAGTTACACCTACTATCCAGACAGTGTGAAGG
GGCGATTCACCATCTCCAGAGACAATGTCAAGAACACCCTGTACCTGCA
AATGAGCAGTCTGAAGTCTGAGGACACAGCCATGTATTACTGTGCAAGA
CAGGGGCTTGATGATAACTACGCGGAGTGGTACTTCGATGTCTGGGGCG
CAGGGACCACGGTCACCGTCTCCTCA

The nucleotide sequence of the variable light chain of the anti-Pra1 antibody produced by the hybridoma cell line 8C3 (Accession number: DSM ACC 3369) is defined by SEQ ID NO: 18:

GACATCCTGCTGACCCAGTCTCCAGCCACCCTGTCTGTGACTCCAGGAG
AAACAGTCAGTCTTTCCTGTAGGGCCAGCCAGAGTATTTACAAGAACCT
ACACTGGTATCAACAGAAATCACATCGGTCTCCAAGGCTTCTCATTAAG
TATGCTTCTGATTCCATCTCTGGGATCCCCTCCAGGTTCACTGGCAGTG
GATCAGGGACAGATTACACTCTCAGTATCAACAGTGTGAAGCCCGAAGA
TGAAGGAAAATATTACTGTCTTCAAGGTTTCAGCACACCGTGGACGTTC
GGTGGAGGCACCAAGCTGGAAATCAAA

Table 4 shows the regions of amino acid sequences defining the CDRs of the anti-TEF1 antibody produced by the hybridoma cell line 5E1 (Accession number: DSM ACC 3368).

CDR-H1, CDR-H2, and CDR-H3 correspond to CDR1, CDR2, and CDR3 respectively, and are located on the heavy variable chain of the antibody.
CDR-L1, CDR-L2, and CDR-L3 correspond to CDR4, CDR5, and CDR6 respectively, and are located on the light variable chain of the antibody.

TABLE 4
Regions of amino acid sequence defining the CDRs
anti-TEF1 antibody 5E1:

| Region | Sequence |
|---|---|
| CDR-H1 | SYAMS (SEQ ID NO: 7) |
| CDR-H2 | SISSGGSTYYPDSVKG (SEQ ID NO: 8) |
| CDR-H3 | GYDGYDY (SEQ ID NO: 9) |
| CDR-L1 | KSSQSLLDSDGKTYLN (SEQ ID NO: 10) |
| CDR-L2 | LVSKLDS (SEQ ID NO: 11) |
| CDR-L3 | WQGTHFPFT (SEQ ID NO: 12) |

The amino acid sequence of the variable heavy chain of the anti-TEF1 antibody produced by the hybridoma cell line 5E1 (Accession number: DSM ACC 3368) is defined by SEQ ID NO: 15:

EVKLVESGGGLVKPGGSLKLSCAASGFTFSSYAMSWVRQTPEKRLEWVA
SISSGGSTYYPDSVKGRFTISRDNARNILYLQMSSLRSEDTAMYYCARG
YDGYDYWGQGTTLTVSS

The CDRs are bolded and are flanked by the framework regions.

The amino acid sequence of the variable light chain of the anti-TEF1 antibody produced by the hybridoma cell line 5E1 (Accession number: DSM ACC 3368) is defined by SEQ ID NO: 16:

DVVMTQTPLTLSVTIGQPASISCKSSQSLLDSDGKTYLNWLLQRPGQSP
KRLIYLVSKLDSGVPDRFTGSGSGTDFTLKISRVEAEDLGVYYCWQGTH
FPFTFGSGTKLEIK

The CDRs are bolded and are flanked by the framework regions.

The nucleotide sequence of the variable heavy chain of the anti-TEF1 antibody produced by the hybridoma cell line 5E1 (Accession number: DSM ACC 3368) is defined by SEQ ID NO: 19:

GAAGTGAAGCTGGTGGAGTCTGGGGGAGGCTTAGTGAAGCCTGGAGGGT
CCCTGAAACTCTCCTGTGCAGCCTCTGGATTCACTTTCAGTAGCTATGC
CATGTCTTGGGTTCGCCAGACTCCAGAGAAGAGGCTGGAGTGGGTCGCA
TCCATTAGTAGTGGTGGTAGCACCTACTATCCAGACAGTGTGAAGGGCC
GATTCACCATCTCCAGAGATAATGCCAGGAACATCCTGTACCTGCAAAT
GAGCAGTCTGAGGTCTGAGGACACGGCCATGTATTACTGTGCAAGAGGC
TATGATGGTTACGACTACTGGGGCCAAGGCACCACTCTCACAGTCTCCT
CA

The nucleotide sequence of the variable light chain of the anti-TEF1 antibody produced by the hybridoma cell line 5E1 (Accession number: DSM ACC 3368) is defined by SEQ ID NO: 20:

GATGTTGTGATGACCCAGACTCCACTCACTTTGTCGGTTACCATTGGAC
AACCAGCCTCCATCTCTTGCAAGTCAAGTCAGAGCCTCTTAGATAGTGA
TGGAAAGACATATTTGAATTGGTTGTTACAGAGGCCAGGCCAGTCTCCA
AAGCGCCTAATCTATCTGGTGTCTAAACTGGACTCTGGAGTCCCTGACA
GGTTCACTGGCAGTGGATCAGGGACAGATTTCACACTGAAAATCAGCAG
AGTGGAGGCTGAGGATTTGGGAGTTTATTATTGCTGGCAAGGTACACAT
TTTCCATTCACGTTCGGCTCGGGGACAAAGTTGGAAATAAAA

### Example 8—Further Characterization of Antibodies of the Invention

The anti-Pra1 antibody produced by the hybridoma cell line 8C3 (Accession number: DSM ACC 3369) has a heavy chain with a mouse IgG₁ isotype and a light chain with a mouse kappa isotype.

The heavy chain comprises a signal peptide defined by amino acid sequence SEQ ID NO: 21, the variable heavy chain defined by amino acid sequence SEQ ID NO: 13, and a constant region defined by amino acid sequence SEQ ID NO: 22.

The signal peptide of the heavy chain is encoded by the nucleotide sequence defined by SEQ ID NO:23. The constant region is encoded by the nucleotide sequence defined by SEQ ID NO: 24.

The light chain comprises a signal peptide defined by amino acid sequence SEQ ID NO: 25, the variable light chain defined by amino acid sequence SEQ ID NO: 14, and a constant region defined by amino acid sequence SEQ ID NO: 26.

The signal peptide of the light chain is encoded by the nucleotide sequence defined by SEQ ID NO:27. The constant region of the light chain is encoded by the nucleotide sequence defined by SEQ ID NO: 28.

The anti-TEF1 antibody produced by the hybridoma cell line 5E1 (Accession number: DSM ACC 3368) has a heavy chain with a mouse IgM isotype and a light chain with a mouse kappa isotype.

The heavy chain comprises a signal peptide defined by amino acid sequence SEQ ID NO: 29, the variable heavy chain defined by amino acid sequence SEQ ID NO: 15, and a constant region defined by amino acid sequence SEQ ID NO: 30.

The signal peptide of the heavy chain is encoded by the nucleotide sequence defined by SEQ ID NO:31. The constant region is encoded by the nucleotide sequence defined by SEQ ID NO: 32.

The light chain comprises a signal peptide defined by amino acid sequence SEQ ID NO: 33, the variable light chain defined by amino acid sequence SEQ ID NO: 16, and a constant region defined by amino acid sequence SEQ ID NO: 34.

The signal peptide of the light chain is encoded by the nucleotide sequence defined by SEQ ID NO:35. The constant region of the light chain is encoded by the nucleotide sequence defined by SEQ ID NO:36.

### Example 9—Further Characterization of the Mode of Action of the Anti-Tef1 Antibodies of the Invention

The impact of mAb 5E1 on complement regulatory activity of its fungal target Tef1 was evaluated.

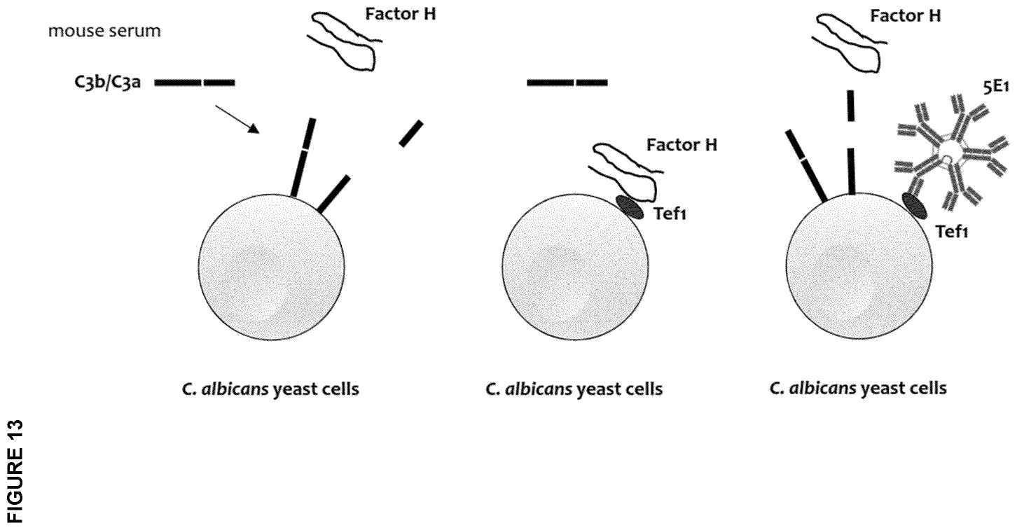

FIG. 13 shows a scheme of how mAb 5E1 reverses the inhibitory effect of Tef1 on complement component C3.

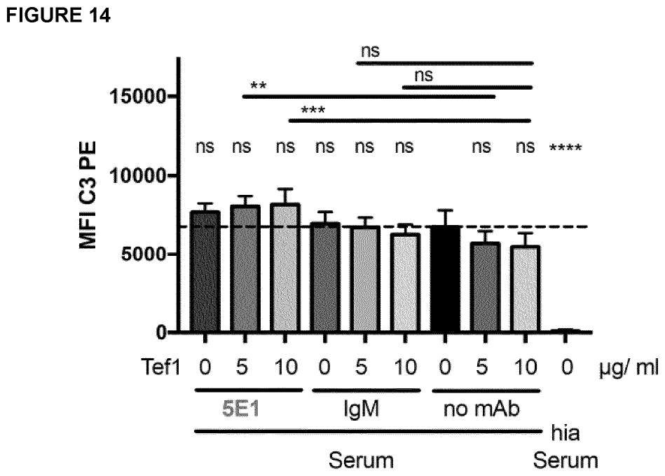

FIG. 14 summarizes data on the blockade of the Tef1 effect on C3 attachment to the surface of C. albicans yeast cells. Concentration of the mAb was 10 μg/ml each; 2% mouse serum was used (n=3-6; One way-ANOVA Tukey's multiple comparison test; ns: not significant; **p<0.01; ***p<0.001; hia: heat-inactivated).

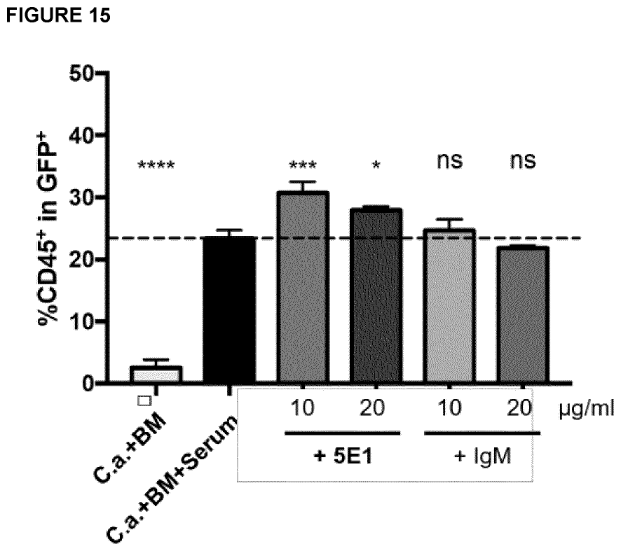

FIG. 15 shows that mAb 5E1 increases phagocytosis of (GFP-transgenic) C. albicans yeast cells by mouse bone marrow cells (n=3; One way-ANOVA Tukey's multiple comparison test; ns: not significant; *p<0.05; **p<0.01; ***p<0.001; ****p<0.0001; C.a.: C. albicans; BM: Bone Marrow).

### REFERENCES

1. Matthews, R. C., G. Rigg, S. Hodgetts, T. Carter, C. Chapman, C. Gregory, C. Illidge, and J. Burnie. 2003. Preclinical assessment of the efficacy of mycograb, a human recombinant antibody against fungal HSP90. Antimicrobial agents and chemotherapy 47:2208-2216.
2. Richie, D. L., M. A. Ghannoum, N. Isham, K. V. Thompson, and N. S. Ryder. 2012. Nonspecific effect of Mycograb on amphotericin B MIC. Antimicrobial agents and chemotherapy 56:3963-3964.
3. Rudkin, F. M., I. Raziunaite, H. Workman, S. Essono, R. Belmonte, D. M. MacCallum, E. M. Johnson, L. M. Silva, A. S. Palma, T. Feizi, A. Jensen, L. P. Erwig, and N. A. R. Gow. 2018. Single human B cell-derived monoclonal anti-Candida antibodies enhance phagocytosis and protect against disseminated candidiasis. Nat Commun 9:5288.
4 Gow, N. A., F. M. Rudkin, and A. Jensen. 2016. Antibody molecules and uses thereof. WO2016142660.
5. Matveev, A. L., V. B. Krylov, Y. A. Khlusevich, I. K. Baykov, D. V. Yashunsky, L. A. Emelyanova, Y. E. Tsvetkov, A. A. Karelin, A. V. Bardashova, S. S. W. Wong, V. Aimanianda, J. P. Latge, N. V. Tikunova, and N. E. Nifantiev. 2019. Novel mouse monoclonal antibodies specifically recognizing beta-(1→3)-D-glucan antigen. PloS one 14: e0215535.
6. Moragues, M. D., M. J. Omaetxebarria, N. Elguezabal, M. J. Sevilla, S. Conti, L. Polonelli, and J. Ponton. 2003. A monoclonal antibody directed against a Candida albicans cell wall mannoprotein exerts three anti-C. albicans activities. Infect Immun 71:5273-5279.
7. Brena, S., M. J. Omaetxebarria, N. Elguezabal, J. Cabezas, M. D. Moragues, and J. Ponton. 2007. Fungicidal monoclonal antibody C7 binds to Candida albicans Als3. Infect Immun 75:3680-3682.

1. Antibody directed against pH regulated antigen 1 (Pra1) of Candida albicans, or an antibody fragment thereof, comprising a complementarity-determining region 1 (CDR1), which comprises an amino acid sequence as defined by SEQ ID NO:1, a complementarity-determining region 2 (CDR2), which comprises an amino acid sequence as defined by SEQ ID NO:2. a complementarity-determining region 3 (CDR3), which comprises an amino acid sequence as defined by SEQ ID NO:3, a complementarity-determining region 4 (CDR4) as defined by SEQ ID NO:4, a complementarity-determining region 5 (CDR5), which comprises an amino acid sequence as defined by SEQ ID NO:5, and a complementarity-determining region 6 (CDR6), which comprises an amino acid sequence as defined by SEQ ID NO:6, wherein any one of the CDR sequences can be altered by substitution, deletion, or insertion of 1 or 2 amino acids.

2. Antibody of claim 1, or an antibody fragment thereof, wherein the antibody is a murine, chimeric, human, or humanized antibody.

3. Antibody of claim 1, wherein the antibody is the monoclonal antibody 8C3 directed against Pra1 of Candida albicans, produced by the hybridoma cell line with the accession number DSM ACC 3369.

4. Hybridoma cell line with Accession number DSM ACC 3369.

5. Antibody directed against translation elongation factor 1 (Tef1) of Candida albicans, or an antibody fragment thereof, comprising a complementarity-determining region 1 (CDR1), which comprises an amino acid sequence as defined by SEQ ID NO:7, a complementarity-determining region 2 (CDR2), which comprises an amino acid sequence as defined by SEQ ID NO:8, a complementarity-determining region 3 (CDR3), which comprises an amino acid sequence as defined by SEQ ID NO:9, a complementarity-determining region 4 (CDR4), which comprises an amino acid sequence as defined by SEQ ID NO:10, a complementarity-determining region 5 (CDR5), which comprises an amino acid sequence as defined by SEQ ID NO: 11, and a complementarity-determining region 6 (CDR6), which comprises an amino acid sequence as defined by SEQ ID NO: 12, wherein any one of the CDR sequences can be altered by substitution, deletion, or insertion of 1 or 2 amino acids.

6. Antibody of claim 5, or an antibody fragment thereof, wherein the antibody is a murine, chimeric, human, or humanized antibody.

7. Antibody of claim 5, wherein the antibody is the monoclonal antibody 5E1 directed against Tef1 of Candida albicans, produced by the hybridoma cell line with the accession number DSM ACC 3368.

8. Hybridoma cell line with Accession number DSM ACC 3368.

9. Pharmaceutical composition comprising the antibody of claim 1, and optionally pharmaceutically acceptable excipients and/or carriers.

10. Pharmaceutical composition comprising the antibody of claim 5, and optionally pharmaceutically acceptable excipients and/or carriers.

11. Antibody of claim 1 or pharmaceutical composition of claim 9 for use in a method of treating a Candida infection in a subject.

12. Antibody of claim 5 or pharmaceutical composition of claim 10 for use in a method of preventing, suppressing, or delaying the emergence of a Candida infection in a subject at risk of acquiring a Candida infection.

13. Antibody of claim 5 or pharmaceutical composition of claim 10 and antibody of any one of claims 1 to 3 or pharmaceutical composition of claim 9 for use in preventing, suppressing or delaying the emergence of a Candida infection in a subject at risk of acquiring a Candida infection and/or in a method of treating a Candida infection in a subject, wherein preventing, suppressing or delaying the emergence of a Candida infection comprises administration of the antibody of claim 5 or 7 or the pharmaceutical composition of claim 10, and wherein treating comprises administration of the antibody of any one of claims 1 to 3 or the pharmaceutical composition of claim 9 if the subject acquires a Candida infection.

14. Antibody or pharmaceutical composition for use of claim 11, wherein preventing, suppressing or delaying the emergence of a Candida infection in a subject at risk of acquiring a Candida infection and/or treating a Candida infection in a subject further comprises administration of an antifungal drug, such as Caspofungin.

15. Antibody or pharmaceutical composition for use of claim 11, wherein the subject at risk of acquiring a Candida infection and/or the subject suffering from a Candida infection is selected from subjects, who have received an organ transplant, or a bone marrow transplantation, subjects in recuperation after extended surgery, subjects on immunosuppressants, subjects diagnosed with HIV, or subjects suffering from COPD (Chronic Obstructive Pulmonary Disease).
